(19) United States
(12) Patent Application Publication
(10) Pub. No.: US 2017/0027726 A1
(43) Pub. Date: Feb. 2, 2017

(54) STENT DELIVERY SYSTEM AND STENT DELIVERY METHOD

(71) Applicant: TERUMO KABUSHIKI KAISHA, Tokyo (JP)

(72) Inventor: Kenji OYAMA, Kanagawa (JP)

(73) Assignee: TERUMO KABUSHIKI KAISHA, Tokyo (JP)

(21) Appl. No.: 15/264,997

(22) Filed: Sep. 14, 2016

Related U.S. Application Data
(63) Continuation of application No. PCT/JP2015/055053, filed on Feb. 23, 2015.

(30) Foreign Application Priority Data
Mar. 17, 2014 (JP) 2014-052928

(51) Int. Cl. A61F 2/966; A61F 2/915

(52) U.S. Cl. CPC A61F 2/966; A61F 2/915; A61F 2002/9517

(57) ABSTRACT

A stent delivery system includes a tube body having a proximal portion latch section which comes into contact with a proximal end of a stent to restrict the movement of the stent in a proximal direction, a stent accommodation unit slidable in the proximal end direction of the tube body while covering a distal side of the tube body and the stent that is to be accommodated inside the stent accommodation unit, and a pulling wire for pulling the stent accommodation unit in the proximal end direction. The stent delivery system further includes an extension restraining wire whose one end portion is fixed to the tube body to restrain the tube body from extending in a distal end direction, a first pulling unit that moves the pulling wire in the proximal end direction, and a second pulling unit that moves the extension restraining wire in the proximal end direction.

### CROSS-REFERENCES TO RELATED APPLICATIONS

This application is a continuation of International Application No. PCT/JP2015/055053, filed on Feb. 23, 2015, and which claims priority to Japanese Patent Application No. 2014-52928, filed on Mar. 17, 2014, the entire contents of both of which are incorporated herein by reference.

### TECHNICAL FIELD

The present invention relates to a stent delivery system in which a stent is caused to indwell a stenosis site or an occluded site appearing in a body lumen so as to maintain a patency state of the lumen, and to a stent delivery method.

### BACKGROUND DISCUSSION

In recent years, for example, in order to treat myocardinal infarction or angina pectoris, a method has been used in which a stent is caused to indwell a lesion area (stenosed site) of a coronary artery so as to secure a space inside the coronary artery. In some cases, in order to treat a stenosed site appearing in other body lumens such as blood vessels, biliary ducts, bronchial tubes, esophagi, and urethrae, a similar method has been used.

The stents are classified as either a balloon-dilatable stent or a self-expandable stent, depending on functions and methods for stent indwelling.

In the case of the balloon-dilatable stent, the stent itself has no expandable function. After the stent is inserted into a target area, the stent is expanded and plastically deformed by a balloon. In this manner, the stent is fixedly and closely attached to the inside of the lumen.

In contrast, in the case of the self-expandable stent, the stent itself has the expandable function. The stent is accommodated in a catheter in a state where the stent has a decreased diameter prior to insertion. After reaching a target area, the stent is expanded by releasing the state having the decreased diameter. In this manner, the stent is fixedly and closely attached to the inside of the lumen.

For example, JP-A-11-313893 discloses a method as follows. The self-expandable stent having the decreased diameter is accommodated inside a tubular stent accommodation unit. A tube body including a stent latch section which can come into contact with the stent is inserted into the stent accommodation unit. In the target area inside the living body lumen, in a state where the stent latch section restricts the movement of the stent in a proximal end direction, the stent accommodation unit is moved in the proximal end direction. In this manner, the stent is forced from the stent accommodation unit so as to expand the stent.

In the above-described stent delivery system, there is a possibility that a phenomenon may occur in which an axial length of a stent caused to indwell a target area becomes shorter than an actual length (shortening).

This phenomenon occurs as follows. An overall stent delivery system is pressed back in a proximal end direction by using a friction force between a stent accommodation unit and the stent, which is generated when the stent is released. Thereafter, the stent is gradually forced from the stent accommodation unit. As a result, a contact area between the stent accommodation unit and the stent decreases, thereby decreasing the friction force. The stent is released while the stent delivery system moves again in a distal end direction.

In particular, when the stent is completely released from the stent accommodation unit, a force for pressing the stent delivery system back in the proximal end direction is eliminated at once. Consequently, the phenomenon in which the length of the stent considerably decreases (jumping) is likely to occur.

The disclosure herein is directed to solving the above-described problem, and is to providing a stent delivery system and a stent delivery method, in which a self-expandable stent is enabled to expand and indwell in a suitable state while a phenomenon of an axially shortened length of the stent is restrained from occurring.

### SUMMARY

According to the disclosure herein, there is provided a stent delivery system including a tube body which has a guide wire lumen, a stent accommodation unit which is slidable in a proximal end direction of the tube body while covering a distal side of the tube body, a substantially cylindrical stent which is expandable radially outward using a self-expandable force by being accommodated inside the stent accommodation unit in a contracted state in a central axis direction and being released from the stent accommodation unit, and a pulling shaft for pulling the stent accommodation unit to the tube body in the proximal end direction by one end portion being fixed to the stent accommodation unit and being moved in the proximal end direction. A stent latch section for restricting a movement of the stent in the proximal end direction by coming into contact with a proximal end of the stent accommodated inside the stent accommodation unit is disposed in the tube body. The stent delivery system includes an extension restraining shaft whose one end portion is fixed to the tube body so as to restrain the tube body from extending in a distal end direction, a first pulling unit that is disposed in a proximal portion of the stent delivery system, and with which a proximal portion of the pulling shaft is interlocked so as to move the pulling shaft in the proximal end direction, and a second pulling unit that is disposed in the proximal portion of the stent delivery system, and with which a proximal portion of the extension restraining shaft is interlocked so as to move the extension restraining shaft in the proximal end direction.

The stent delivery system configured as described above has the extension restraining shaft that restrains the tube body from extending in the distal end direction. Accordingly, the extension restraining shaft can stop a force which causes the tube body in a state of being flexed in the proximal end direction to extend again in the distal end direction. When the stent is released, the tube body is restrained from extending in the distal end direction. Therefore, the stent is allowed to expand and indwell in a suitable state while the likelihood of a shortened length of the stent occurring is reduced.

The stent delivery system may have an interlocking unit that interlocks and operates the first pulling unit and the second pulling unit, and that can release the interlocking therebetween. In this case, workability is improved by interlocking and operating the first pulling unit and the second pulling unit. In a case where only one unit is intended to be operated or in a case where the two units are intended to be separately operated, the interlocking therebetween can be released.

In a state where the first pulling unit and the second pulling unit are interlocked with each other by the interlocking unit, a movement amount of the extension restraining shaft moved by the second pulling unit in the proximal end direction may be equal to or smaller than a movement amount of the pulling shaft moved by the first pulling unit in the proximal end direction. In this case, a movement of a stent latch section in the proximal end direction is equal to or smaller than a length in which the stent is intended to move. Accordingly, the stent latch section is not separated from the stent. When the stent is released, the stent latch section can satisfactorily restrict the movement of the stent, and the stent can be suitably released.

The first pulling unit may have a first movement restriction unit that restricts the movement of the pulling shaft in the distal end direction, and the second pulling unit may have a second movement restriction unit that restricts the movement of the extension restraining shaft in the distal end direction. In this case, it is possible to satisfactorily maintain a state where the first pulling unit and the second pulling unit are moved in the proximal end direction, thereby improving operability.

In addition, according to the disclosure, there is provided a stent delivery method for delivering a stent by using a stent delivery system including a tube body which has a guide wire lumen, a stent accommodation unit which is slidable in a proximal end direction of the tube body while covering a distal side of the tube body, a substantially cylindrical stent which is expandable radially outward using a self-expandable force by being accommodated inside the stent accommodation unit in a contracted state in a central axis direction and being released from the stent accommodation unit, and a pulling shaft for pulling the stent accommodation unit to the tube body in the proximal end direction by one end portion being fixed to the stent accommodation unit and being moved in the proximal end direction. A stent latch section for restricting a movement of the stent in the proximal end direction by coming into contact with a proximal end of the stent accommodated inside the stent accommodation unit is disposed in the tube body. The stent delivery method includes a first step of causing the pulling shaft to move the stent accommodation unit in the proximal end direction, while causing the stent latch section to restrict the movement of the stent in the proximal end direction, a second step of causing an extension restraining shaft whose one end portion is fixed to the tube body to pull the tube body in the proximal end direction concurrently with or after the first step so as to apply a tensile force to a site fixed to the tube body in the proximal end direction, and a third step of causing the pulling shaft to move the stent accommodation unit in the proximal end direction, after the second step, and expanding the stent by using a self-expandable force after releasing the stent in the distal end direction of the stent accommodation unit, while causing the stent latch section to restrict the movement of the stent in the proximal end direction.

According to the stent delivery method configured as described above, in the second step, the tube body is pulled in the proximal end direction by the extension restraining shaft, and the tensile force is applied to a site fixed to the tube body in the proximal end direction. Accordingly, the extension restraining shaft can stop a force which causes the tube body in a state of being deflected in the proximal end direction to extend again in the distal end direction.

Therefore, in the third step, when the stent is released, the tube body can be restrained from extending in the distal end direction. Therefore, the stent is enabled to expand and indwell in a suitable state while the phenomenon of a shortened length of the stent is less likely to occur.

The stent delivery method may further include performing the second step concurrently with the first step by interlocking and operating a first pulling unit that is disposed in a proximal portion of the stent delivery system, and with which a proximal portion of the pulling shaft is interlocked so as to move the pulling shaft in the proximal end direction, and a second pulling unit that is disposed in the proximal portion of the stent delivery system, and with which a proximal portion of the extension restraining shaft is interlocked so as to move the extension restraining shaft in the proximal end direction, and a step of releasing the interlocking between the first pulling unit and the second pulling unit after the second step. In this case, workability can be improved by interlocking and operating the first pulling unit and the second pulling unit. In a case where only one unit is intended to be operated or in a case where the two units are intended to be separately operated, the interlocking therebetween can be released.

A movement amount of the pulling shaft pulled in the proximal end direction by the first pulling unit may be equal to or larger than a movement amount of the extension restraining shaft pulled in the proximal end direction by the second pulling unit. In this case, a movement of a stent latch section in the proximal end direction is equal to or smaller than a length in which the stent is intended to move. Accordingly, the stent latch section is not separated from the stent. When the stent is released, the stent latch section can satisfactorily restrict the movement of the stent, and the stent can be suitably released.

### BRIEF DESCRIPTION OF THE DRAWINGS

FIG. 1 is a plan view illustrating a stent delivery system according to an exemplary embodiment of the disclosure herein.

FIG. 2 is a sectional view illustrating a distal portion of the stent delivery system according to the exemplary embodiment.

FIG. 3 is a sectional view illustrating the distal portion of the stent delivery system according to the exemplary embodiment.

FIG. 4 is a view for describing an internal structure for the operation unit of the stent delivery system according to the exemplary embodiment.

FIG. 5 is a partially cutout sectional view for describing the internal structure of the operation unit of the stent delivery system according to the exemplary embodiment.

FIG. 6 is a plan view illustrating a state where a self-expandable stent expands.

FIG. 7 is a deployment view illustrating a state where a diameter of the self-expandable stent is reduced.

FIG. 8 is view for describing an example when the stent delivery system according to the exemplary embodiment is inserted into a body lumen.

FIG. 9 is a sectional view illustrating a distal portion of the stent delivery system when the stent delivery system according to the exemplary embodiment is inserted into the body lumen.

FIG. 10 is a view for describing a state where a rotary roller for operating the stent delivery system according to the exemplary embodiment is pressed.

FIG. 11 is a view for describing a state of interlocking a first pulling unit and a second pulling unit of the stent delivery system according to the exemplary embodiment.

FIG. 12 is a sectional view illustrating the distal portion of the stent delivery system when the rotary roller for operation is rotated in a state of interlocking the first pulling unit and the second pulling unit of the stent delivery system according to the exemplary embodiment.

FIG. 13 is a view for describing a state of releasing the interlocking between the first pulling unit and the second pulling unit of the stent delivery system according to the exemplary embodiment.

FIG. 14 is a sectional view illustrating the distal portion of the stent delivery system when the stent delivery system according to the exemplary embodiment causes a stent to indwell.

FIG. 15 is a view for describing an internal structure of an operation unit according to a modified example of the stent delivery system according to the exemplary embodiment.

### DETAILED DESCRIPTION

### Description of Embodiments

Hereinafter, an exemplary embodiment according to the disclosure herein will be described with reference to the drawings.

In some cases, dimensional proportions in the drawings may be exaggerated and different from actual proportions for convenience of description.

A stent delivery system 1 according to the exemplary embodiment of the disclosure causes a stent 3 to indwell a stenosed site or an occluded site appearing inside blood vessels, biliary ducts, bronchial tubes, esophagi, urethrae, or other body lumens so as to maintain a patency state of the lumen.

In this description, a side inserted into the lumen is referred to as a “distal end” or a “distal side”, and an operating hand side is referred to as a “proximal end” or a “proximal side”.

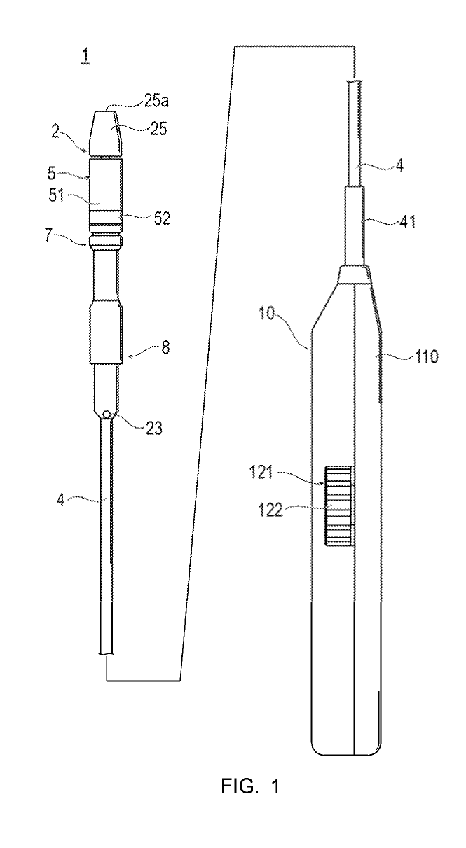

As illustrated in FIGS. 1 to 3, the stent delivery system 1 according to the exemplary embodiment includes a tube body which has a guide wire lumen 21, a cylindrical stent accommodation unit 5 which is slidable in a proximal end direction of the tube body while covering a distal side of the tube body, a stent 3 which is accommodated inside the stent accommodation unit 5, a slide tube 7 which is arranged in a proximal end of the stent accommodation unit 5, and a pulling wire 6 (pulling shaft) whose distal portion is fixed to the stent accommodation unit 5 in order to pull the stent accommodation unit 5, an extension restraining wire 9 (extension restraining shaft) whose distal portion is fixed to the tube body in order to apply a tensile force to the tube body, and an operation unit 10 which is disposed in a proximal portion of the stent delivery system 1 in order to perform operations.

The tube body includes a distal side tube 2 which has the guide wire lumen 21, a proximal side tube 4 whose distal portion is fixed to a proximal end portion of the distal side tube 2, and a fixing tube 8 including an opening 23 to which the proximal portion of the distal side tube 2 and the distal portion of the proximal side tube 4 are fixed, and which communicates with the guide wire lumen 21.

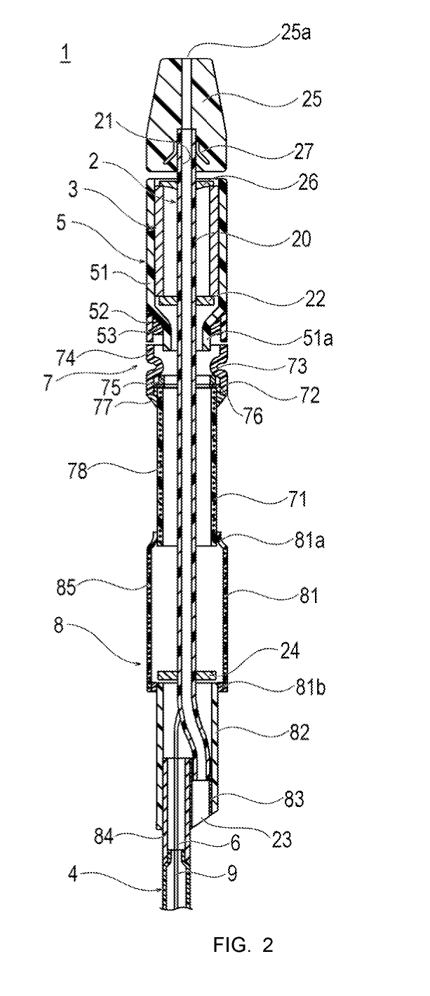

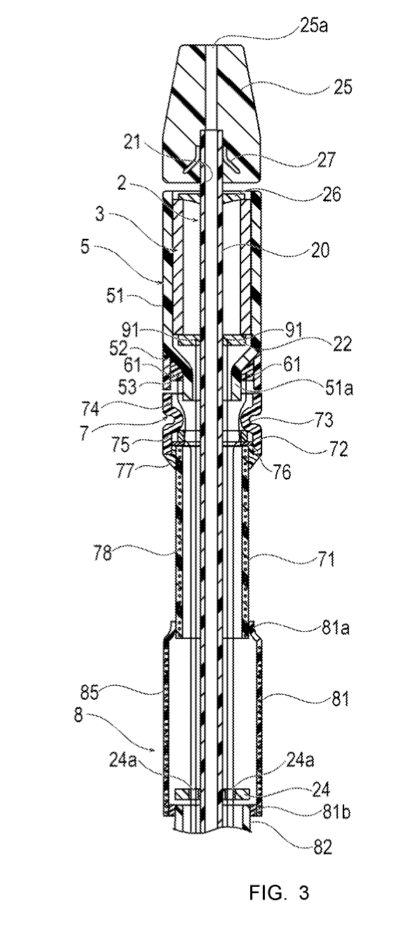

As illustrated in FIGS. 2 and 3, the distal side tube 2 includes a distal side tube main body 20 serving as a tube body having the guide wire lumen 21 extending from a distal end to a proximal end, a distal member 25 which is fixed to the distal end of the distal side tube main body 20, a stent distal portion latch section 26 for latching the distal portion of the stent 3, and a stent proximal portion latch section 22 (stent latch section) for latching the proximal portion of the stent 3.

In the distal side tube 2, the outer diameter is 0.3 to 2.0 mm, and preferably 0.5 to 1.5 mm. The inner diameter is 0.2 to 1.5 mm, and preferably 0.3 to 1.2 mm. The length is 20 to 600 mm, and preferably 30 to 450 mm.

As illustrated in FIGS. 1 to 3, the distal member 25 forms a distal portion of the distal side tube 2, and a distal opening portion 25a in which the guide wire lumen 21 is open is formed in the distal end of the distal member 25.

Alternatively, the distal member 25 may be formed integrally with the distal side tube main body 20.

A proximal portion of the distal side tube main body 20 is fixed to the fixing tube 8. In addition, the guide wire lumen 21 of the distal side tube main body 20 communicates with an opening 23 disposed in a fixing tube 8.

It is preferable that the distal member 25 is located on the distal side from the distal end of the stent accommodation unit 5 and is formed in a tapered shape whose diameter gradually decreases toward the distal end. In this manner, the distal member 25 is easily inserted into a stenosed site. In addition, the distal member 25 also functions as a stopper which prevents the stent accommodation unit 5 from moving in the distal end direction.

The outer diameter of the most distal portion of the distal member 25 is preferably 0.5 mm to 1.8 mm.

In addition, the outer diameter of the largest diameter portion of the distal member 25 is preferably 0.8 to 4.0 mm. Furthermore, the length of the distal side tapered portion is preferably 2.0 to 20.0 mm.

The stent proximal portion latch section 22 (stent latch section) restricts the movement of the stent to the proximal side of the stent 3. As illustrated in FIGS. 2 and 3, the stent proximal portion latch section 22 is disposed at a position on the proximal side away from the distal end of the distal side tube 2 as far as a predetermined distance.

It is preferable that the stent proximal portion latch section 22 is an annular projection portion which projects radially outward. Thus, the distal side from the stent proximal portion latch section 22 serves as a stent accommodation portion.

The outer diameter of the stent proximal portion latch section 22 is configured to have a size which can come into contact with the proximal end of the contracted stent 3. Hence, even if the stent accommodation unit 5 moves to the proximal side, the stent proximal portion latch section 22 maintains the position of the stent 3. Consequently, the stent 3 is released from the stent accommodation unit 5.

The stent distal portion latch section 26 restricts the movement of the stent 3 to the distal side. As illustrated in FIGS. 2 and 3, the stent distal portion latch section 26 is disposed at a position on the distal side away from the stent proximal portion latch section 22 as far as a predetermined distance (substantially the axial length of the stent 3).

It is preferable that the stent distal portion latch section 26 is an annular projection portion which projects radially outward. The stent distal portion latch section 26 is located on the proximal side slightly from the distal end of the stent accommodation unit 5. It is preferable that the stent distal portion latch section 26 is the annular projection portion. Thus, a stent accommodation portion is disposed between the stent distal portion latch section 26 and the stent proximal portion latch section 22.

The outer diameter of the stent distal portion latch section 26 has a size which can come into contact with the distal end of the contracted stent 3.

In addition, the stent distal portion latch section 26 has a tapered surface in which the diameter of the proximal surface decreases in the proximal end direction.

Therefore, when the stent 3 is released, the stent distal portion latch section 26 does not hinder the stent 3. In addition, it becomes easy to collect (specifically, accommodate inside a guiding catheter or a sheath) the stent delivery system 1 after the stent 3 is released.

The outer diameter of the stent proximal portion latch section 22 and the stent distal portion latch section 26 is preferably 0.8 to 4.0 mm.

The stent proximal portion latch section 22 and the stent distal portion latch section 26 are preferably the annular projection portions as illustrated. However, as long as the movement of the stent 3 can be restricted and released, any configuration may be adopted. For example, both of these may be one or multiple projections disposed integrally with the distal side tube 2 or by using a separate member.

In addition, the stent proximal portion latch section 22 and the stent distal portion latch section 26 may be formed of a separate member having an X-ray contrast material. In this manner, it is possible to accurately recognize the position of the stent 3 by using X-ray contrast, thereby further facilitating manual skills. For example, as the X-ray contrast material, it is preferable to use gold, platinum, a platinum-iridium alloy, silver, stainless steel, platinum, or an alloy thereof. Then, the stent proximal portion latch section 22 and the stent distal portion latch section 26 form a wire by using the X-ray contrast material so as to be wound around the outer surface of the distal side tube 2. Alternatively, both latch sections form a pipe by using the X-ray contrast material so as to be attached by means of caulking or bonding.

A slide tube latch section 24 restricts the movement of the slide tube 7 to the proximal side. As illustrated in FIGS. 2 and 3, the slide tube latch section 24 is fixed to the outer peripheral surface of the distal side tube main body 20 inside the fixing tube 8.

It is preferable that the slide tube latch section 24 is an annular projection portion which projects radially outward.

In addition, as illustrated in FIG. 3, a passage 24a through which the pulling wire 6 and the extension restraining wire 9 pass is formed in the slide tube latch section 24.

The outer diameter of the slide tube latch section 24 has a size which can come into contact with the proximal end of the slide tube 7 moving inside the fixing tube 8 in the proximal end direction.

As a material for forming the distal side tube main body 20, it is preferable to use a material which is rigid and flexible to some extent. For example, it is possible to preferably use polyolefin such as polyethylene and polypropylene, polyester such as polyamide, polyethylene terephthalate, fluorine-based polymer such as ETFE, polyether ether ketone (PEEK), or polyimide. In particular, among the above-described resins, it is preferable to use a thermoplastic resin.

An outer surface from which the distal side tube 2 is exposed may be coated with a biocompatible resin, particularly antithrombotic resin. For example, as an antithrombotic material, it is preferable to use poly-hydroxyethyl methacrylate and a copolymer of hydroxyethyl methacrylate and styrene (for example, HEMA-St-HEMA block copolymer).

In a case where the distal member 25 is configured to include a material separate or different from that of the distal side tube main body 20, as the distal member 25, it is preferable to use a flexible material.

For example, synthetic resin elastomer such as olefin-based elastomer (for example, polyethylene elastomer, polypropylene elastomer), polyamide elastomer, styrene elastomer (for example, styrene-butadiene-styrene copolymer, styrene-isoprene-styrene copolymer, styrene-ethylene butylene-styrene copolymer), polyurethane, urethane system elastomer, fluorine resin-based elastomer, and rubber-like materials including synthetic rubber such as urethane rubber, silicone rubber, butadiene rubber, and natural rubber such as latex rubber are used.

In particular, in the stent delivery system 1 according to the exemplary embodiment, the distal side tube main body 20 and the distal member 25 are formed as separate members, and a stopper member 27 is fixed to the distal portion of the distal side tube main body 20.

The stopper member 27 includes a cylindrical portion fixed to the distal side tube 2, and a skirt portion which spreads in a tapered shape from the cylindrical portion. Then, the stopper member 27 is thus in a state of being embedded into the distal member 25, thereby preventing the distal member 25 from being detached and moving to the distal side. It is preferable to form the stopper member 27 by using metal (for example, stainless steel).

As illustrated in FIGS. 1 to 3, the fixing tube 8 includes a distal side fixing tube 81 having a large outer diameter, and a proximal side fixing tube 82 fixed to the proximal portion of the distal side fixing tube 81.

The distal side fixing tube 81 includes a distal end decreased diameter portion 81a. An inner surface of the distal end decreased diameter portion 81a is in contact with an outer peripheral surface of the proximal portion of the slide tube 7.

The slide tube 7 is not fixed to the distal side fixing tube 81 and may therefore slide to the proximal side. In this manner, the slide tube 7 can be accommodated inside the distal side fixing tube 81 after entering the inside of the distal side fixing tube 81.

As illustrated in FIGS. 2 and 3, the distal portion of the proximal side fixing tube 82 is fixed by a fixing portion 81b after entering the inside of the proximal end of the distal side fixing tube 81.

The distal side fixing tube 81 includes a reinforcement layer 85 over substantially the entire distal side fixing tube 81.

As the reinforcement layer, it is preferable to use a layer which has a mesh shape or a spiral shape. In particular, it is preferable that the reinforcement layer has the mesh shape. As the mesh-shaped reinforcement layer, it is preferable to use those which are formed in the mesh shape by using a thin metal wire. As the thin metal wire, it is preferable to use stainless steel. Furthermore, it is preferable that no reinforcement layer is present in a portion configured to be connected to the proximal side fixing tube 82.

A tubular fixing member 83 which accommodates the proximal portion of the distal side tube 2 is disposed in the proximal portion of the distal side tube 2. In addition, the tubular fixing member 84 is disposed in the distal end of the proximal side tube 4. The tubular fixing member 83 and the tubular fixing member 84 are fixedly attached to the proximal side fixing tube 82.

As illustrated in FIGS. 1 to 3, the proximal side tube 4 serves as a tube body extending from the distal end to the proximal end, and the operation unit 10 is fixed to the proximal end of the proximal side tube 4.

The distal portion of the proximal side tube 4 is bonded to the fixing tube 8 by the tubular fixing member 84.

The proximal side tube 4 internally includes a pulling wire lumen into which the pulling wire 6 and the extension restraining wire 9 can be inserted.

For the proximal side tube 4, the length is 300 mm to 1,500 mm, and preferably 1,000 to 1,300 mm. The outer diameter is 0.5 to 1.5 mm, and preferably 0.6 to 1.3 mm. The inner diameter is 0.3 to 1.4 mm, and preferably 0.5 to 1.2 mm.

A shifted angle between a central axis of the proximal side tube 4 and a central axis of the distal side tube main body 20 is preferably 0.1 to 2.0 mm, and particularly preferable 0.5 to 1.5 mm.

As a forming material of the proximal side tube 4, it is preferable to use a material which is rigid and flexible. For example, it is possible to preferably use polyolefin such as polyethylene and polypropylene, fluorine-based polymer such as nylon, polyethylene terephthalate, and ETFE, polyether ether ketone (PEEK), or polyimide.

An outer surface of the proximal side tube may be coated with a biocompatible resin, particularly antithrombotic resin. For example, as an antithrombotic material, it is preferable to use poly-hydroxyethyl methacrylate and a copolymer of hydroxyethyl methacrylate and styrene (for example, HEMA-St-HEMA block copolymer).

In addition, as a forming material of the proximal side tube 4, it is preferable to use a relatively rigid material. For example, metal such as Ni—Ti, brass, stainless steel, and aluminum can be used. Further, it is also possible to use a relatively rigid resin, for example, such as polyimide, vinyl chloride, and polycarbonate.

As illustrated in FIGS. 2 and 3, the stent accommodation unit 5 covers the distal side of the distal side tube 2, and is slidable in the proximal end direction of the distal side tube 2.

The stent accommodation unit 5 serves as a tube body provided with a predetermined length, and the distal end and the proximal end are open.

The distal opening portion functions as a releasing port of the stent 3 when the stent 3 is caused to indwell a stenosed site inside the lumen.

As illustrated in FIG. 14, the stent 3 is ejected from the distal end portion, thereby releasing a stress load. In this manner, the stent 3 expands, and the stent 3 is restored to a shape formed before being compressed.

It is preferable that the stent accommodation unit 5 has the length of approximately 20 mm to 400 mm. It is particularly preferable that the stent accommodation unit 5 has the length of 30 mm to 300 mm. In addition, the outer diameter of the stent accommodation unit 5 is preferably set to approximately 1.0 to 4.0 mm. It is particularly preferable to set the outer diameter to 1.5 to 3.0 mm. In addition, the inner diameter of the stent accommodation unit 5 is preferably set to approximately 1.0 to 2.5 mm.

The stent accommodation unit 5 includes a tubular member main body portion 51 including a small-diameter portion 51a disposed in the proximal portion, and a tubular portion 52 disposed so as to cover the small-diameter portion 51a.

The proximal portion of the small-diameter portion 51a projects from the tubular portion 52 in the proximal end direction. The tubular portion 52 is fixed to the proximal portion of the tubular member main body portion 51. Then, a fixing point 61 serving as the distal portion of the pulling wire 6 enters a gap formed between the small-diameter portion 51a and the tubular portion 52, and is fixed to the stent accommodation unit 5 by using a fixing agent 53 filling the gap. The tubular member main body portion 51 and the tubular portion 52 are integrated with each other by the fixing agent 53.

The small-diameter portion 51a includes a tapered portion whose diameter decreases toward the proximal side, and a short cylindrical portion which extends to the proximal side from the tapered portion.

The tubular portion 52 which covers the small-diameter portion 51a of the tubular member main body portion 51 is fixed to the proximal portion of the tubular member main body portion 51. As the fixing agent, it is preferable to use an adhesive such as an epoxy resin, an ultraviolet curing resin, and a cyanoacrylate resin. However, a thermal fusing method may be employed.

In the stent accommodation unit 5 according to the exemplary embodiment, the tubular member main body portion 51 and the tubular portion 52 except for the small-diameter portion 51a have substantially the same outer diameter.

The outer diameter of the stent accommodation portion of the tubular member main body portion 51 is preferably set to approximately 1.0 to 4.0 mm. It is particularly preferable to set the outer diameter to 1.5 to 3.0 mm.

In addition, the length of the stent accommodation unit 5 is preferably set to approximately 20 to 400 mm. It is particularly preferable to set the length to 30 mm to 300 mm.

Further, the length of the tubular member main body portion 51 is preferably set to approximately 10 to 200 mm. It is particularly preferable to set the length to 15 mm to 150 mm. The length of the tubular portion 52 is preferably set to approximately 10 to 200 mm. It is particularly preferable to set the length to 15 mm to 150 mm.

Without being limited to embodiments which include the tubular member main body portion 51 and the proximal side tubular portion 52 which are as described above, the stent accommodation unit 5 may be an integrated member.

As illustrated in FIGS. 2 and 3, the slide tube 7 together with the stent accommodation unit 5 is pulled by the pulling wire 6 so as to be movable in the proximal end direction. The slide tube 7 is not fixed to the stent accommodation unit 5.

The slide tube 7 includes a slide tube main body 71 and a distal side tubular member 72 which is fixed to the distal portion of the slide tube main body 71, which covers the distal end of the slide tube main body 71, and which extends to the distal side of the stent delivery system 1 from the distal end of the slide tube main body 71.

The distal side tubular member 72 is an integrally molded tube body having a decreased diameter portion 73 which is located between the distal end and the proximal end of the distal side tubular member 72 and in which at least the inner diameter is reduced.

The slide tube 7 is arranged so that the distal end is close to the proximal end of the stent accommodation unit 5.

In addition, the slide tube 7 can be accommodated inside the fixing tube 8 when inserted from the proximal side.

The slide tube 7 may adopt a structure in which the slide tube 7 is covered with the fixing tube 8 from the proximal side.

The inner diameter of the decreased diameter portion 73 is substantially the same as, slightly larger than, or slightly smaller than the inner diameter of the slide tube main body 71.

Furthermore, in the distal side tubular member 72, the outer diameter and the inner diameter of the portions other than at least the decreased diameter portion 73 are larger than those of the slide tube main body 71.

The decreased diameter portion 73 is arranged between the distal end and the proximal end of the distal side tubular member 72.

A ring-shaped member 75 is accommodated between the distal end of the slide tube main body 71 and the decreased diameter portion 73 of the distal side tubular member 72. The pulling wire 6 is fixed to the ring-shaped member 75.

The inner diameter of the decreased diameter portion 73 of the distal side tubular member 72 is larger than the outer diameter of the distal side tube main body 20. Therefore, the distal side tubular member 72 is movable to the proximal side without coming into contact with the distal side tube main body 20.

In addition, the inner diameter of the decreased diameter portion 73 of the distal side tubular member 72 is smaller than the outer diameter of the ring-shaped member 75. Therefore, the decreased diameter portion 73 restricts the movement of the ring-shaped member 75 in the distal end direction.

The slide tube 7 is pulled to the proximal side by the pulling wire 6, thereby moving to the proximal side together with the ring-shaped member 75.

In addition, the ring-shaped member 75 is not fixed to either the slide tube main body 71 or the distal side tubular member 72, and is rotatably accommodated between the distal end of the slide tube main body 71 and the decreased diameter portion 73 of the distal side tubular member 72.

However, the movement of the ring-shaped member 75 in the axial direction inside the slide tube 7 is not possible except for a clearance.

As the ring-shaped member 75, it is preferable to use a metal ring.

It is preferable to fix the pulling wire 6 to the ring-shaped member 75 by welding or by using an adhesive.

The distal side tubular member 72 of the slide tube 7 allows the rotary movement of the ring-shaped member 75, and the decreased diameter portion 73 and the distal end of the slide tube main body 71 substantially hinder large movements in the axial direction of the ring-shaped member 75.

In this way, the ring-shaped member 75 is rotatable with respect to the slide tube 7. Accordingly, the ring-shaped member 75, the fixing portion of the pulling wire, and the pulling wire itself are less likely to follow the rotary movement of the distal side tubular member 72 (slide tube 7).

In addition, a resin ring 76 may be arranged between the ring-shaped member 75 and the distal end of the slide tube main body 71. The resin ring 76 is arranged in this manner, thereby further facilitating the rotary movement of the ring-shaped member 75.

As the resin ring 76, it is preferable to use those which have less frictional resistance.

As the resin ring, it is possible to use a fluorine-based polymer such as ETFE, polyether ether ketone (PEEK), or polyamide.

In addition, the proximal portion of the distal side tubular member 72 is fixed to the distal portion of the slide tube main body 71 by an adhesive 77. The adhesive 77 may be prevented from entering the ring-shaped member 75 by arranging the resin ring 76 between the ring-shaped member 75 and the distal end of the slide tube main body 71.

In addition, in the distal side tubular member 72 of the slide tube 7, it is preferable that a distal portion 74 thereof covers the proximal portion of the small-diameter portion 51a of the stent accommodation unit 5.

In addition, it is preferable that the distal side tubular member 72 of the slide tube 7 and the stent accommodation unit 5 are not bonded together.

The distal side tubular member 72 and the stent accommodation unit 5 are not substantially in contact with each other, and the distal portion of the distal side tubular member 72 of the slide tube 7 covers the proximal portion of the small-diameter portion 51a of the stent accommodation unit 5.

Furthermore, the slide tube main body 71 includes a reinforcement layer 78 over the entire body of the slide tube main body 71.

The reinforcement layer is disposed in this way, thereby improving kink resistance and providing satisfactory sliding ability of the slide tube 7.

It is preferable that the reinforcement layer is a mesh-shaped reinforcement layer.

It is preferable to form the mesh-shaped reinforcement layer by using a blade wire.

For example, the mesh-shaped reinforcement layer can be formed by using a wire blade such as metal wires made of stainless steel, elastic metal, super-elastic alloy, or shape memory alloy, in which the wire diameter is 0.01 to 0.2 mm and preferably 0.03 to 0.1 mm.

Alternatively, the mesh-shaped reinforcement layer may be formed by using synthetic fibers such as polyamide fibers, polyester fibers, and polypropylene fibers.

As a forming material of the stent accommodation unit 5 (the tubular member main body portion 51 and the tubular portion 52), the slide tube 7 (the slide tube main body 71 and the distal side tubular member 72), and the fixing tube 8 (the distal side fixing tube 81 and the proximal side fixing tube 82), in view of physical properties required for these members or tubes (flexibility, rigidity, strength, sliding ability, kink resistance, and elasticity), for example, it is preferable to use fluorine-based polymer such as polyethylene, polypropylene, nylon, polyethylene terephthalate, polyimide, PTFE, and ETFE. A thermoplastic elastomer is more preferable.

The thermoplastic elastomer is appropriately selected from nylon-based elastomer (for example, polyamide elastomer), urethane-based elastomer (for example, polyurethane elastomer), polyester-based elastomer (for example, polyethylene terephthalate elastomer), and olefin-based elastomer (for example, polyethylene elastomer or polypropylene elastomer).

Furthermore, it is preferable that the outer surface of the stent accommodation unit 5 is subjected to processing for lubricating the outer surface.

For example, this processing includes a coating or fixing of a hydrophilic polymer such as poly hydroxyethyl methacrylate, poly hydroxyethyl acrylate, hydroxypropyl cellulose, methyl vinyl ether-maleic anhydride copolymer, polyethylene glycol, polyacrylamide, and polyvinyl pyrrolidone.

In addition, in order to provide satisfactory sliding ability for the stent 3, the above-described materials may coat the inner surface of the stent accommodation unit 5 or may be fixed thereto.

In addition, the stent accommodation unit 5 may be formed in combination by a two-layer structure made of the above-described polymers (for example, the outer surface is formed of nylon and the inner surface is formed of PTFE).

As illustrated in FIGS. 2 to 5, the pulling wire 6 is disposed at one or more locations (two locations in the present exemplary embodiment).

In the pulling wire 6, in a gap portion included in the above-described stent accommodation unit 5, the fixing point 61 is fixed to the outer side of the small-diameter portion 51a of the stent accommodation unit 5 by the fixing agent 53.

Thus, the pulling wire 6 extends in the proximal end direction from the fixing point 61 across the proximal end of the stent accommodation unit 5, and extends through the slide tube 7, the fixing tube 8, and the proximal side tube 4.

Hence, when the pulling wire 6 is pulled in the proximal end direction, it thereby causes the stent accommodation unit 5 and the slide tube 7 to move in the proximal end direction.

Furthermore, as described above, the pulling wire 6 is also fixed to the ring-shaped member 75 included in the slide tube 7.

Therefore, in the stent delivery system 1, the pulling wire 6 is pulled in the proximal end direction. Accordingly, the ring-shaped member 75 is also pulled to the proximal side, and the slide tube 7 comes into contact with the ring-shaped member 75. In this manner, the slide tube is also pulled to the proximal side.

Therefore, a structure is adopted in which the stent accommodation unit 5 and the slide tube 7 are respectively and individually pulled. Accordingly, when being pulled, the stent accommodation unit 5 and the slide tube 7 do not come into contact with each other.

In addition, a force applied when the pulling wire 6 is pulled is dispersed to the fixing point 61 and a fixing portion of the ring-shaped member 75 which is a member moved by pulling, thereby reliably preventing unfixing between the pulling wire 6 and the stent accommodation unit 5 in the fixing point 61.

The extension restraining wire 9 is a member for restraining the tube body from extending in the distal end direction. As illustrated in FIGS. 2 to 5, the extension restraining wire 9 extends from the operation unit 10, and passes through the proximal side tube 4, the slide tube 7, and the stent accommodation unit 5. A distal end 91 is fixed to the stent proximal portion latch section 22.

The distal end 91 of the extension restraining wire 9 is fixed by being embedded into a forming material of the stent proximal portion latch section 22.

Alternatively, the distal end 91 of the extension restraining wire 9 may be fixed to the stent proximal portion latch section 22 by welding or by using an adhesive.

For both the pulling wire 6 and the extension restraining wire 9, it is possible to preferably use a wire rod or multiple twisted wire rods.

In addition, a size of the pulling wire 6 and the extension restraining wire 9 is not particularly limited. Normally, it is preferable to set the size to approximately 0.01 to 1.5 mm, and more preferable to set the size to approximately 0.1 to 1.0 mm.

As a configuration material of the pulling wire 6 and the extension restraining wire 9, it is possible to preferably use a wire rod or multiple twisted wire rods.

In addition, the wire diameter of the pulling wire is not particularly limited. Normally, it is preferable to set the wire diameter to approximately 0.01 to 0.55 mm, and more preferable to set the wire diameter to approximately 0.1 to 0.3 mm.

In addition, a forming material of the pulling wire 6 and the extension restraining wire 9 includes a stainless steel wire (preferably, high-tensile stainless steel for spring), a piano wire (preferably, piano wire subjected to nickel-plating or chrome-plating), or a super elastic alloy wire, wire rods formed of various types of metal such as Ni—Ti alloy, Cu—Zn alloy, Ni—Al alloy, tungsten, tungsten alloy, titanium, titanium alloy, cobalt alloy, and tantalum, a relatively high rigid polymer material such as polyamide, polyimide, ultra-high molecular weight polyethylene, polypropylene, and fluorine resin, or an appropriate combination of these materials.

Further, a side surface of the pulling wire 6 and the extension restraining wire 9 may be coated with a low-friction resin for increasing lubricity.

The low-friction resin includes a fluorine-based resin, nylon 66, polyether ether ketone, high-density polyethylene.

Among these materials, it is more preferable to use the fluorine-based resin.

For example, the fluorine-based resin includes polytetrafluoroethylene, polyvinylidene fluoride, ethylene tetrafluoroethylene, and perfluoroalkoxy resins.

In addition, the coating may be performed using silicone or various hydrophilic resins.

The stent 3 is formed in a substantially cylindrical shape. As illustrated in FIGS. 2 and 3, in a state where the stent 3 is compressed in the central axis direction, the stent 3 is accommodated inside the stent accommodation unit 5. When being released from the stent accommodation unit 5, the stent 3 expands radially outward, and is restored to a shape formed before being compressed.

As long as the stent 3 is a so-called self-expandable stent, any type may be employed.

For example, as illustrated in FIG. 6, in a state where the stent 3 expands and is restored to the shape before being compressed, multiple annular portions 31 formed in an annular shape while being bent are arrayed side by side in the axial direction. The annular portions 31 adjacent to each other in the axial direction are connected by a connection portion 32, thereby configuring substantially one cylindrical shape.

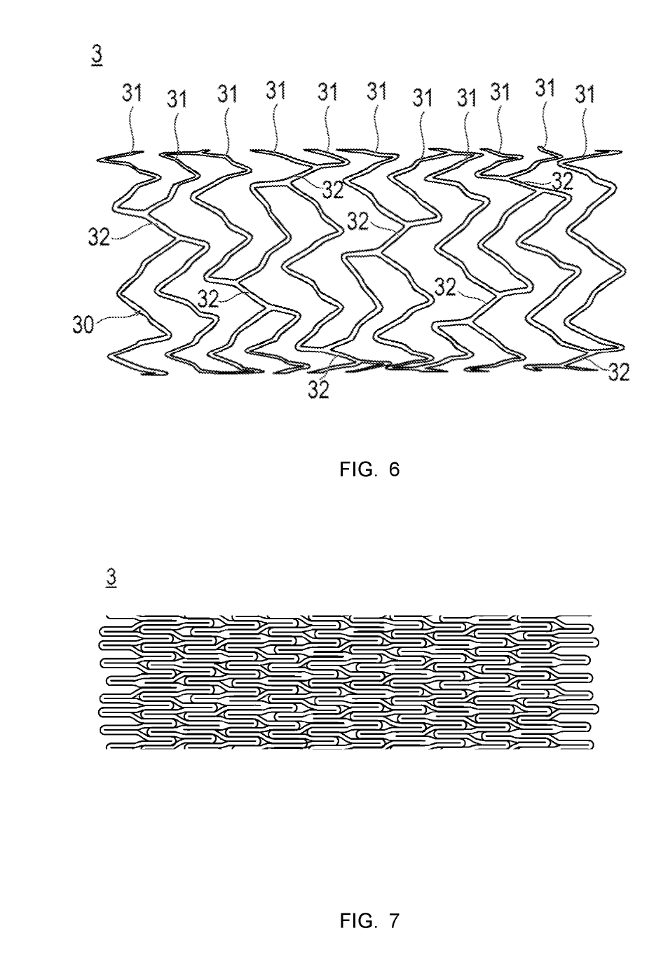

As in the deployment view illustrated in FIG. 7, in a state where the stent 3 is compressed in the central axis direction, the stent 3 is accommodated inside the stent accommodation unit 5.

For example, the stent 3 is manufactured as follows. A super-elastic alloy pipe (to be described later) having an outer diameter suitable for an indwelling-targeted site inside a living body is prepared. A side surface of the pipe is partially removed by means of cutting (for example, mechanical cutting or laser cutting) or chemical etching. Multiple cutout portions or multiple openings are formed on the side surface.

In a state where the stent 3 expands, the outer diameter of the stent 3 is 2.0 to 30 mm, and preferably 2.5 to 20 mm. The inner diameter is 1.4 to 29 mm, and preferably 1.6 to 28 mm. The length in the axial direction is 10 to 150 mm, and preferably 15 to 100 mm.

A shape of the stent 3 is not particularly limited to the shape illustrated in FIGS. 6 and 7.

That is, as long as the shape of the stent 3 can be decreased when being inserted and can be increased (restored) when being released from the inside of the body, the shape is not limited to the above-described shape.

For example, any shape may be employed such as a coil shape, a cylindrical shape, a roll shape, a deformed pipe shape, a supercoil shape, a plate spring coil shape, a basket shape or a mesh shape.

As a forming material of the stent 3, a super-elastic alloy is preferably used.

The super-elastic alloy described herein is generally called a shape memory alloy, and represents those which are at least super-elastic at a biological temperature (approximately, 37□C).

Particularly, super-elastic metal such as Ti—Ni alloy containing Ni of 49 to 53 atom %, Cu—Zn alloy containing Zn of 38.5 to 41.5 wt %, Cu—Zn—X alloy (X═Be, Si, Sn, Al, and Ga) containing X of 1 to 10 wt %, or Ni—Al alloy containing Al of 36 to 38 atomic % is preferably used.

It is particularly preferable to use the above-described Ti—Ni alloy.

In addition, Ti—Ni alloy is partially substituted with X of 0.01 to 10.0% X so as to form Ti—Ni—X alloy (X═Co, Fe, Mn, Cr, V, Al, Nb, W, and B). Alternatively, Ti—Ni alloy is partially substituted with 0.01 to 30.0 atom % so as to form Ti—Ni—X alloy (X═Cu, Pb, and Zr). Alternatively, a cold working ratio and/or a condition for final heat treatment is selected. In this manner, the mechanical properties can be appropriately changed.

In addition, the mechanical properties can be appropriately changed by using the above-described Ti—Ni—X alloy and by selecting the cold working ratio and/or the condition for final heat treatment.

As illustrated in FIGS. 1, 4, and 5, the operation unit 10 includes a housing 110, a first pulling unit 120 for performing a pulling operation of the pulling wire 6, a second pulling unit 130 for pulling the extension restraining wire 9, and a sealing mechanism 140.

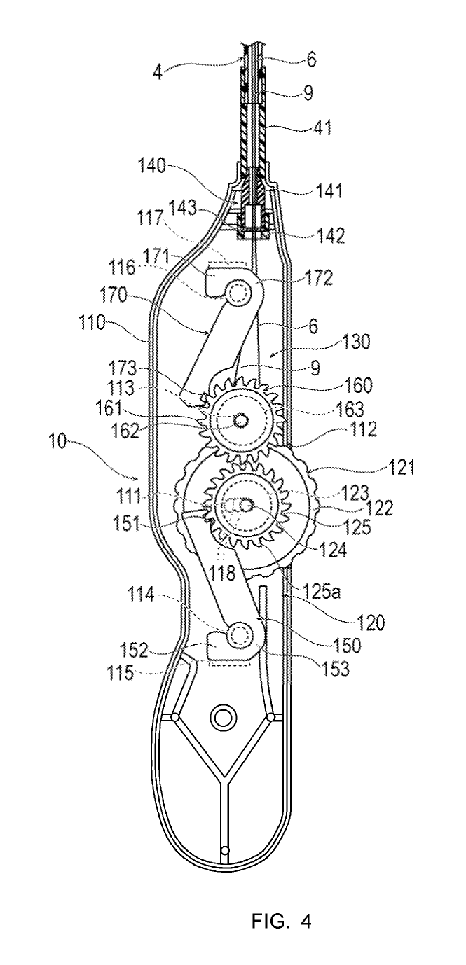

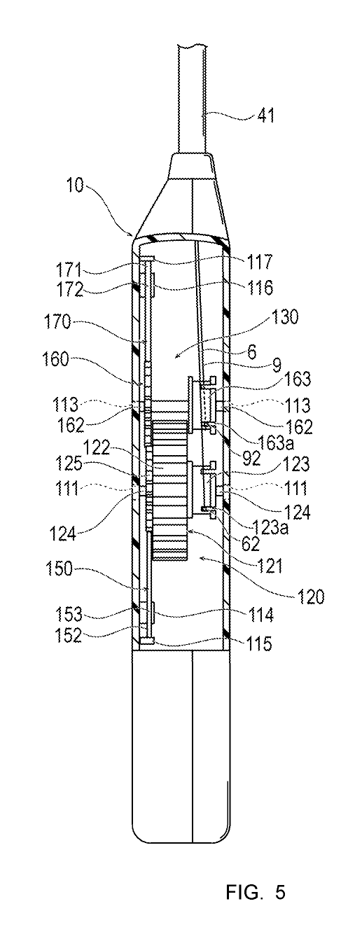

As illustrated in FIGS. 4 and 5, the first pulling unit 120 includes an operation rotary roller 121 for performing a pulling operation of the pulling wire 6, and a first biasing member 150.

The operation rotary roller 121 includes a roller portion 122 operated by an operator, a first winding shaft portion 123 for winding the pulling wire 6, a first rotary shaft 124, and a first gear portion 125.

The roller portion 122, which is rotatably operated by the operator, is formed in a disc shape, and is arranged inside the housing 110 so as to be partially exposed from the housing 110.

A portion of the roller portion 122, which is exposed from the housing 110, is operated by the operator.

It is preferable that a front surface portion which tends to be touched by the operator when the roller portion 122 is operated is configured to include a non-slip front surface.

For example, it is preferable that the outer peripheral surface of the roller portion 122 is subjected to knurling processing, embossing processing, or high friction material coating.

The first rotary shaft 124 is formed coaxially with the roller portion 122 while projecting from both side surfaces of the roller portion 122.

The first rotary shaft 124 is rotatably accommodated in a groove-shaped first bearing portion 111 formed in the housing 110, and is movable along a groove inside the first bearing portion 111.

Since the first rotary shaft 124 is movable along the groove inside the first bearing portion 111, the operator presses the operation rotary roller 121 through an opening portion 112 of the housing 110. In this manner, the operation rotary roller 121 is movable in a direction in which the operation rotary roller 121 is pressed into the housing 110.

The first winding shaft portion 123 holds the proximal portion of the pulling wire 6, and winds the pulling wire 6. The first winding shaft portion 123 is disposed coaxially and integrally with the roller portion 122, and is formed so as to have a smaller diameter than that of the roller portion 122.

A slit 123a which can accommodate the pulling wire 6 is formed in the first winding shaft portion 123. An anchor portion 62 formed in the proximal portion of the pulling wire 6 so as to have a large diameter is accommodated in the slit 123a.

A method of fixing the pulling wire 6 to the first winding shaft portion 123 is not limited to the above-described method.

It is preferable that the proximal portion for winding the pulling wire 6 is flexible in order to facilitate the winding.

As a method of obtaining the flexible proximal portion, it is possible to employ a method of forming the proximal portion of the pulling wire 6 by using a flexible material and a method of decreasing the diameter of the proximal portion of the pulling wire 6.

The first winding shaft portion 123 is disposed coaxially and integrally with the roller portion 122. Accordingly, the roller portion 122 is rotatably rotated and the first winding shaft portion 123 is rotated together with the roller portion 122. In this manner, the pulling wire 6 can be wound around the outer peripheral surface of the first winding shaft portion 123.

It is preferable that a winding amount of the pulling wire 6 is smaller than a rotary operation amount of the roller portion 122.

In this manner, the pulling wire 6 can be slowly wound. The stent accommodation unit 5 can be slowly moved to the proximal side. While a state of the stent 3 is checked, the stent 3 can be suitably released from the stent accommodation unit 5.

According to the exemplary embodiment, the outer diameter of the first winding shaft portion 123 is smaller than that of the roller portion 122. Accordingly, the winding amount of the pulling wire 6 is smaller than the rotary operation amount of the roller portion 122.

It is preferable to set the outer diameter of the first winding shaft portion 123 to approximately 1 to 60 mm, and it is particularly preferable to set the outer diameter to 3 to 30 mm. It is preferable to set the outer diameter of the roller portion 122 to approximately 1 to 20 times the outer diameter of the first winding shaft portion 123, and it is particularly preferable to set the outer diameter to 1 to 10 times.

In addition, it is preferable to set the outer diameter of the roller portion 122 to approximately 10 to 60 mm, and it is particularly preferable to set the outer diameter to 15 to 50 mm.

Without being limited to this integral configuration, the roller portion 122 and the first winding shaft portion 123 may be configured to include a separate member which is rotated in accordance with the rotation of the roller portion 122.

As a rotation transmission system of the roller portion 122, a gear type or a belt type may be employed.

The first gear portion 125 is disposed on a surface opposite to a surface of the roller portion 122, on which the first winding shaft portion 123 is disposed. On the outer peripheral surface of the first gear portion 125, multiple teeth 125a are formed side by side in the circumferential direction.

In the respective teeth 125a of the first gear portion 125, a tilting angle with respect to the outer peripheral surface of one side tooth face is larger than a tilting angle of the other side tooth face.

The first gear portion 125 is disposed coaxially and integrally with the roller portion 122.

The first gear portion 125 meshes with a second gear portion 160 (to be described later), and meshes with a first meshing portion 151 of the first biasing member 150.

Therefore, the second gear portion 160 is rotated in accordance with the rotation of the first gear portion 125.

The first gear portion 125 and the second gear portion 160 function as an interlocking unit which interlocks and operates the first pulling unit 120 and the second pulling unit 130.

The first gear portion 125 has a smaller diameter than that of the roller portion 122. It is preferable to set the outer diameter of the first gear portion 125 to approximately 10 to 60 mm, and it is particularly preferable to set the outer diameter to 15 to 50 mm. It is preferable to set the number of teeth to approximately 4 to 200, and it is particularly preferable to set the number of teeth to 4 to 70.

The first biasing member 150 is arranged inside the housing 110, biases the operation rotary roller 121 in a direction toward the opening portion 112 of the housing 110, and restricts the rotation of the operation rotary roller 121.

The first biasing member 150 includes a first mounting portion 152 which is mounted on and fixed to the housing 110, a first elastically deformable portion 153 which is elastically deformed so as to generate a biasing force, and the first meshing portion 151 which can mesh with the first gear portion 125 while being formed on a side opposite to the first mounting portion 152 across the first elastically deformable portion 153.

The first meshing portion 151 comes into contact with the first gear portion 125, and biases the first gear portion 125 in the direction toward the opening portion 112 of the housing 110 by using a biasing force generated by the first elastically deformable portion 153.

In addition, when the first gear portion 125 is intended to rotate to one side, the first meshing portion 151 allows the rotation of the first gear portion 125 by coming into contact with the tooth face having the small tilting angle. When the first gear portion 125 is intended to rotate to the opposite side, the first meshing portion 151 restricts the rotation of the first gear portion 125 by coming into contact with the tooth face having the large tilting angle.

A direction in which the first gear portion 125 is rotatable is coincident with a direction in which the pulling wire 6 can be wound by the first winding shaft portion 123, and thus, the rotation is restricted in the winding direction and the opposite direction.

Therefore, the first biasing member 150 and the first gear portion 125 function as the first movement restriction unit which restricts the movement direction of the pulling wire 6.

As illustrated in FIGS. 4 and 5, the second pulling unit 130 includes the second gear portion 160 which can mesh with the first gear portion 125, a second rotary shaft 162, a second winding shaft portion 163 which winds the extension restraining wire 9, and a second biasing member 170.

The second gear portion 160 is arranged at a position for meshing with the first gear portion 125 in an initial state where the operation rotary roller 121 is biased in the direction toward the opening portion 112 of the housing 110 by the first biasing member 150.

In respective teeth 161 of the second gear portion 160, the tilting angle with respect to the outer peripheral surface of one side tooth face of a tooth is larger than the tilting angle of the other side tooth face of the tooth.

It is preferable to set the outer diameter of the second gear portion 160 to approximately 10 to 60 mm, and it is particularly preferable to set the outer diameter to 15 to 50 mm. It is preferable to set the number of teeth to approximately 4 to 200, and it is particularly preferable to set the number of teeth to 4 to 70.

The second rotary shaft 162 is formed coaxially with the second gear portion 160 while projecting from both side surfaces of the second gear portion 160.

The second rotary shaft 162 is rotatably accommodated in a second bearing portion 113 formed in the housing 110.

The second winding shaft portion 163 holds the proximal portion of the extension restraining wire 9, and winds the extension restraining wire 9. The second winding shaft portion 163 is disposed coaxially and integrally with the second gear portion 160.

A slit 163a which can accommodate the extension restraining wire 9 is formed in the second winding shaft portion 163. An anchor portion 92 formed in the proximal portion of the extension restraining wire 9 so as to have a large diameter is accommodated in the slit 163a.

A method of fixing the extension restraining wire 9 to the second winding shaft portion 163 is not limited to the above-described method.

If the second gear portion 160 is rotated, the second winding shaft portion 163 is rotated, and the extension restraining wire 9 is wound around the outer peripheral surface of the second winding shaft portion 163.

It is preferable that a winding amount (movement amount) of the extension restraining wire 9 wound by the second winding shaft portion 163 is equal to or smaller than a winding amount (movement amount) of the pulling wire 6 wound by the first winding shaft portion 123.

It is preferable that the proximal portion for winding the extension restraining wire 9 is flexible in order to facilitate the winding.

As a method of obtaining the flexible proximal portion, it is possible to employ a method of forming the proximal portion of the extension restraining wire 9 by using a flexible material and decreasing the diameter of the proximal portion of the extension restraining wire 9.

It is preferable to set the outer diameter of the second winding shaft portion 163 to approximately 1 to 60 mm, and it is particularly preferable to set the outer diameter to 3 to 30 mm.

The second biasing member 170 is arranged inside the housing 110, and restricts the rotation of the second gear portion 160.

The second biasing member 170 includes a second mounting portion 171 which is mounted on and fixed to the housing 110, a second elastically deformable portion 172 which is elastically deformed so as to generate a biasing force, and a second meshing portion 173 which can mesh with the second gear portion 160 while being formed on a side opposite to the second mounting portion 171 across the second elastically deformable portion 172.

The second meshing portion 173 is pressed against the second gear portion 160 due to the biasing force generated by the second elastically deformable portion 172.

In addition, when the second gear portion 160 is intended to rotate to one side, the second meshing portion 173 allows the rotation of the second gear portion 160 by coming into contact with the tooth face having the smaller tilting angle. When the second gear portion 160 is intended to rotate to the opposite side, the second meshing portion 173 restricts the rotation of the second gear portion 160 by coming into contact with the tooth face having the larger tilting angle.

A direction in which the second gear portion 160 is rotatable is coincident with a direction in which the extension restraining wire 9 can be wound by the second winding shaft portion 163, and thus, the rotation is restricted in the winding direction and the opposite direction.

In addition, the direction in which the second gear portion 160 is rotatable is opposite to the direction in which the first gear portion 125 is rotatable.

In this manner, the rotation force of the first gear portion 125 enables the second gear portion 160 to rotate in the opposite direction in accordance with the rotation of the first gear portion 125.

The second biasing member 170 and the second gear portion 160 thus function as a second movement restriction unit which restricts the movement direction of the extension restraining wire 9.

As illustrated in FIGS. 1, 4, and 5, the housing 110 has a shape whose proximal side is bent and rounded, and is easily gripped, thereby facilitating the operation of the operation rotary roller 121 in a gripped state.

A tubular connector 41 is fixed to the distal portion of the housing 110, and the proximal portion of the proximal side tube 4 is fixed to the distal portion of the tubular connector 41.

As illustrated in FIGS. 4 and 5, the housing 110 includes a locking rib (not illustrated) which engages with the teeth 125a of the first gear portion 125, the opening portion 112 for partially exposing the roller portion 122, the first bearing portion 111 which accommodates the first rotary shaft 124, the second bearing portion 113 which accommodates the second rotary shaft 162, a first strut 114 and a first projection portion 115 for fixing the first biasing member 150, and a second strut 116 and a second projection portion 117 for fixing the second biasing member 170.

The locking rib has a shape which can enter a portion between the teeth 125a formed in the first gear portion 125 of the operation rotary roller 121.

The first bearing portion 111 rotatably accommodates the first rotary shaft 124 of the operation rotary roller 121, and has a groove shape extending in a direction away from the above-described opening portion 112.

Without being limited to the groove shape, the first bearing portion 111 may have any shape as long as the first rotary shaft 124 is rotatable and movable.

For example, the shape of the first bearing portion 111 may be an oval shape, a rectangular shape, or an elliptical shape.

In addition, two sets of two ribs 118 which face each other while forming a pair are formed on inner surfaces (inner wall surfaces) facing each other in a concave portion of the first bearing portion 111 extending in a groove shape.

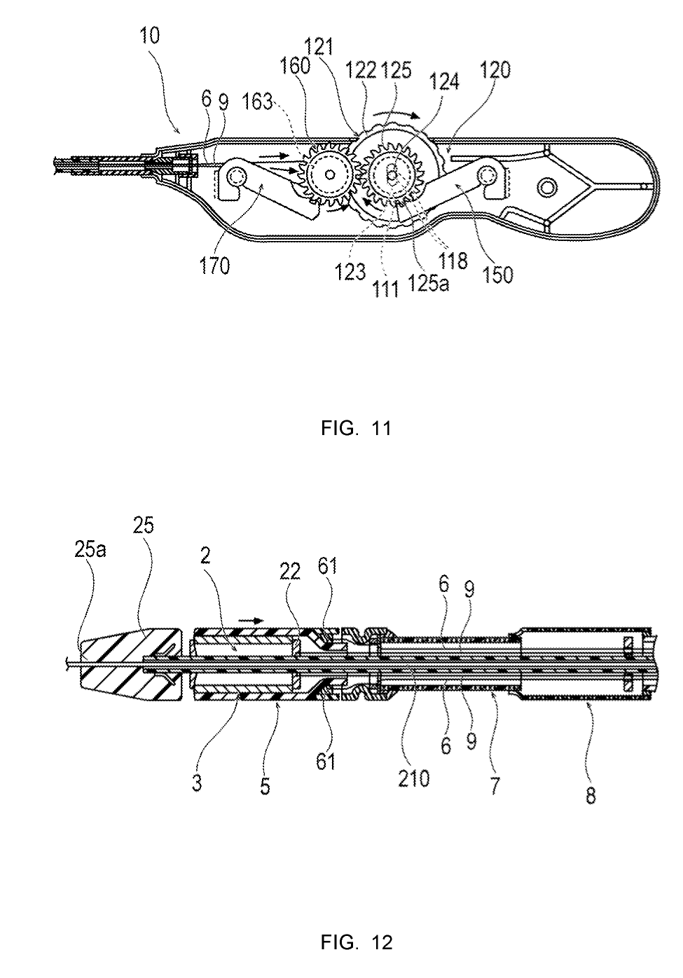

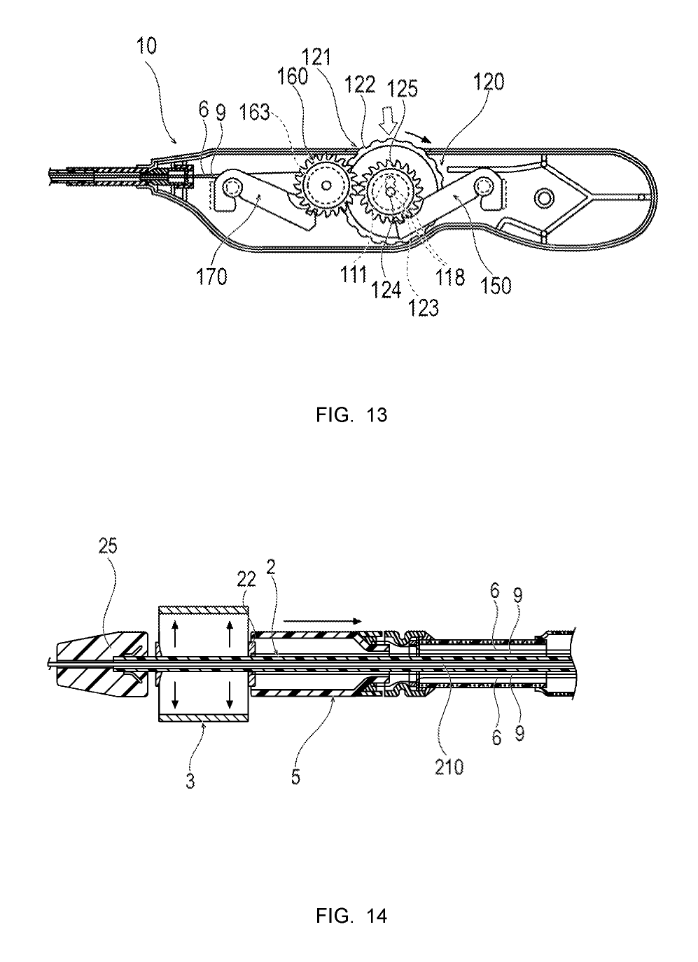

The first rotary shaft 124 is moved so as to ride across the two sets of ribs 118 sequentially. In this manner, it is possible to sequentially switch modes to an initial state where the operation rotary roller 121 is not rotatable (refer to FIGS. 4 and 10), a state where the operation rotary roller 121 is rotatable and the pulling wire 6 and the extension restraining wire 9 can be pulled (refer to FIG. 11), and a state where the operation rotary roller 121 is rotatable and the pulling wire 6 and only the pulling wire 6 can be pulled (refer to FIG. 13).

As illustrated in FIGS. 4 and 5, the second bearing portion 113 rotatably accommodates the second rotary shaft 162 of the second gear portion 160.

The first strut 114 is arranged inside the first elastically deformable portion 153 of the first biasing member 150, and is formed in a cylindrical shape having an outer surface corresponding to an inner surface shape of the first elastically deformable portion 153.

The first projection portion 115 is formed in a plate shape, i.e., planar and generally round.

The first mounting portion 152 of the first biasing member 150 has a shape that can be mounted on a portion between the first strut 114 and the first projection portion 115 which are formed in the housing 110.

The second strut 116 is arranged inside the second elastically deformable portion 172 of the second biasing member 170, and is formed in a cylindrical shape having an outer surface corresponding to an inner surface shape of the second elastically deformable portion 172.

The second projection portion 117 is also formed in a plate shape, i.e., planar and generally round.

The second mounting portion 171 of the second biasing member 170 has a shape that can be mounted on a portion between the second strut 116 and the second projection portion 117 which are formed in the housing 110.

As illustrated in FIG. 4, the sealing mechanism 140 allows the pulling wire 6 and the extension restraining wire 9 which extend from the proximal side tube 4 and the tubular connector 41 in the proximal end direction to move in the axial direction, and maintains a liquid-tight state thereof so as to introduce the pulling wire 6 and the extension restraining wire 9 into the housing 110.

The sealing mechanism 140 includes a tubular main body member 141 including the distal portion to be fixed to the rear end portion of the tubular connector 41, a cap member 142 fixed to the proximal end of the tubular main body member 141, and a sealing member 143 arranged between the tubular main body member 141 and the cap member 142.

The tubular main body member 141 and the cap member 142 include an opening portion allowing the pulling wire 6 and extension restraining wire 9 to extend therethrough.

The sealing member 143 includes a hole portion or a slit for allowing the pulling wire 6 and the extension restraining wire 9 to penetrate in a liquid-tight and slidable state.

As described above, if the operation rotary roller 121 is pressed in the initial state where the operation rotary roller 121 is not rotatable (refer to FIGS. 4 and 10), the first rotary shaft 124 rides across the first set of ribs 118 of the first bearing portion 111. The first biasing member 150 is deflected, and the operation rotary roller 121 is moved, thereby maintaining a state where the first gear portion 125 and the second gear portion 160 mesh with each other. The locking rib is separated from a portion between the teeth formed in the first gear portion 125, thereby enabling the operation rotary roller 121 to be rotated (refer to FIG. 11).

However, the operation rotary roller 121 is only rotatable in the winding direction in which the pulling wire 6 can be wound. If the operation rotary roller 121 is rotated in the direction opposite to the winding direction, the tooth face on which one tooth 125a of the first gear portion 125 has a large tilting angle engages with the first meshing portion 151 of the first biasing member 150, thereby hindering the rotation.

This restricts the rotation of the operation rotary roller 121 in the direction opposite to the winding direction of the pulling wire 6.

Then, if the operation rotary roller 121 is further pressed, the first rotary shaft 124 rides across the second set of ribs 118 of the first bearing portion 111. The first biasing member 150 is deflected, and the operation rotary roller 121 is moved, thereby separating the first gear portion 125 from the second gear portion 160 and releasing a meshing state therebetween. Accordingly, the rotation force of the first gear portion 125 is not transmitted to the second gear portion 160 (refer to FIG. 13).

Next, a method of using the stent delivery system 1 according to the exemplary embodiment of the disclosure will be described.

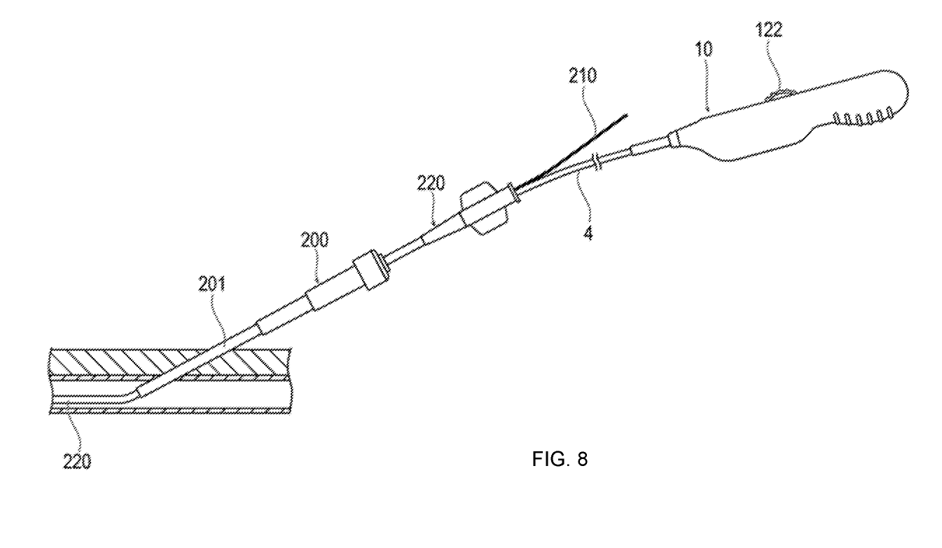

At first, a catheter introducer 200 (refer to FIG. 8) using a Seldinger technique percutaneously punctures a blood vessel.

Subsequently, a guiding catheter 220 having a guide wire 210 inserted into a lumen is inserted into the catheter introducer 200. The guide wire 210 is preceded to the guiding catheter 220, and the distal end of the guiding catheter 220 is inserted into the blood vessel through a distal opening of a sheath 201 of the catheter introducer 200.

Thereafter, while the guide wire 210 is precededly moved, the guiding catheter 220 is gradually pressed ahead so as to reach a target area.

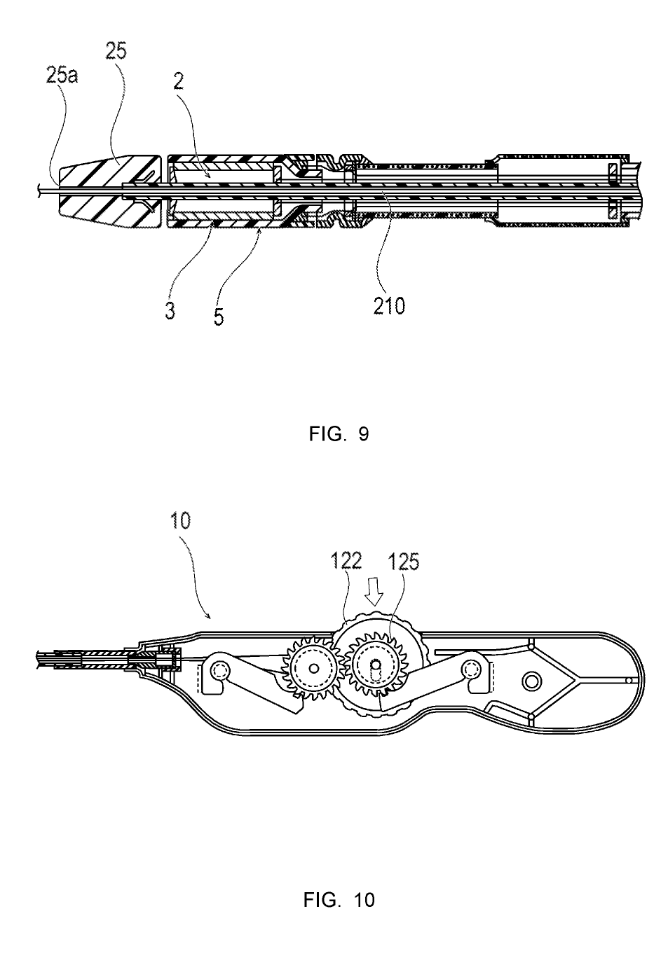

Next, as illustrated in FIG. 9, a terminal end of the guide wire 210 is inserted into the distal opening portion 25a of the distal member 25 of the stent delivery system 1 so as to pull out the guide wire 210 from the opening 23.

Next, as illustrated in FIG. 8, the stent delivery system 1 is inserted into the guiding catheter 220 inserted into a living body, from the distal member 25. The stent delivery system 1 is pressed ahead along the guide wire 210 so as to project from the guiding catheter 220. The stent accommodation portion of the stent accommodation unit 5 is located inside a target stenosed site.

As illustrated in FIG. 10, in an initial state, the operation unit 10 cannot rotate the roller portion 122, since a locking rib (not illustrated) engages with the teeth formed in the first gear portion 125.

Therefore, it is possible to prevent an erroneous operation of the roller portion 122.

Next, if the roller portion 122 of the operation unit 10 is pressed, as illustrated in FIG. 11, the first rotary shaft 124 rides across the first set of ribs 118 of the first bearing portion 111. In turn, the roller portion 122 is moved, and the locking rib is separated from a portion between the teeth 125a formed in the first gear portion 125, thereby enabling the operation rotary roller 121 to be rotated.

In this state, if the roller portion 122 is rotated in the winding direction, the first winding shaft portion 123 of the first pulling unit 120 is rotated, and the pulling wire 6 is wound around the outer peripheral surface of the first winding shaft portion 123. The distal portion of the pulling wire 6 is moved in the proximal end direction (first step).

The first biasing member 150 restricts the rotation of the first winding shaft portion 123 in the direction opposite to the winding direction. Accordingly, it is possible to satisfactorily maintain the winding state of the pulling wire 6.

In addition, if the operation rotary roller 121 is rotated, the second gear portion 160 meshing with the first gear portion 125 is also rotated. The extension restraining wire 9 is wound around the outer peripheral surface of the second winding shaft portion 163 disposed coaxially with the second gear portion 160. The distal portion of the extension restraining wire 9 is thereby moved in the proximal end direction (second step).

As illustrated in FIG. 11, the second biasing member 170 restricts the rotation of the second winding shaft portion 163 in the direction opposite to the winding direction. Accordingly, it is possible to satisfactorily maintain the winding state of the extension restraining wire 9.

As the pulling wire 6 is wound, as illustrated in FIG. 12, the fixing point 61 of the pulling wire 6 is fixed to the stent accommodation unit 5. Accordingly, the stent accommodation unit 5 and the slide tube 7 are moved in the proximal end direction.

Further, since the distal end 91 of the extension restraining wire 9 is fixed to the stent proximal portion latch section 22, if the extension restraining wire 9 is wound, the distal portion of the tube body is moved in the proximal end direction.

At this time, the rear end surface of the stent 3 comes into contact with and is latched by the distal surface of the stent proximal portion latch section 22 of the distal side tube 2. Accordingly, in accordance with the movement of the stent accommodation unit 5, the stent 3 is moved to the stent accommodation unit 5 in the distal end direction.

Then, due to the frictional force generated between the stent accommodation unit 5 and the stent 3 when the stent 3 is released, the tube body including the distal side tube 2, the proximal side tube 4, and the fixing tube 8 is deflected and pressed back in the proximal end direction. The length corresponding to the tube body pressed back is wound by the extension restraining wire 9.

In this case, the winding amount (movement amount) of the extension restraining wire 9 wound by the second winding shaft portion 163 is equal to or smaller than the winding amount (movement amount) of the pulling wire 6 wound by the first winding shaft portion 123. Accordingly, the movement amount of the stent proximal portion latch section 22 in the proximal end direction is equal to or smaller than the length in which the stent 3 is intended to move. Therefore, the stent proximal portion latch section 22 is not released from a state of being in contact with the proximal end of the stent 3.

Hence, when the stent 3 is released, the stent proximal portion latch section 22 satisfactorily restricts the movement of the stent 3, and the stent 3 can be suitably released.

It is possible to have a change in the deflected tube body that causes a change in the proximal end direction, but the position of the stent proximal portion latch section 22 is not changed. In this state, an operator readjusts the position of the stent accommodation unit 5 to a stenosed site in accordance with a change in the length of the stent delivery system 1. If the roller portion 122 of the operation unit 10 is further pressed, as illustrated in FIG. 13, the first rotary shaft 124 rides across the second set of ribs 118 of the first bearing portion 111. The first biasing member 150 is deflected, and the operation rotary roller 121 is moved, thereby separating the first gear portion 125 from the second gear portion 160 and releasing a meshing state therebetween. Accordingly, the rotation force of the first gear portion 125 is not transmitted to the second gear portion 160.

In this manner, the second gear portion 160 is not rotatable. The extension restraining wire 9 applies the tensile force acting in the proximal end direction to a portion having the stent proximal portion latch section 22 of the tube body in deflected state, thereby maintaining a state of stopping the force by which the tube body is intended to extend again in the distal end direction.

Next, if the roller portion 122 of the operation unit 10 is rotated in the winding direction, the second winding shaft portion 163 is not rotated. Accordingly, the extension restraining wire 9 is not wound and only the pulling wire 6 is wound by the first winding shaft portion 123. The stent accommodation unit 5 and the slide tube 7 are moved to the proximal side along the axial direction.

At this time, the rear end surface of the stent 3 comes into contact with and is latched by the distal surface of the stent proximal portion latch section 22 of the distal side tube 2. Accordingly, in accordance with the movement of the stent accommodation unit 5, the stent 3 is released from the distal opening of the stent accommodation unit 5 (third step).

As illustrated in FIG. 14, the stent 3 released in this way is subjected to self-expanding, thereby widening a stenosed site. The stent 3 is caused to indwell the stenosed site.

Incidentally, when the stent 3 is released, the stent 3 is gradually expelled from the stent accommodation unit 5. Consequently, a contact area decreases between the stent accommodation unit 5 and the stent 3, thereby decreasing the frictional force and generating a force by which the tube body in a deflected state is intended to extend again in the distal end direction.

In particular, when the stent 3 is completely expelled from the stent accommodation unit 5, a force for pressing the tube body back in the proximal end direction is eliminated at once. Consequently, a phenomenon in which the length of the stent 3 considerably decreases (jumping) is likely to occur.

However, according to the exemplary embodiment, the extension restraining wire 9 maintains a state of stopping the force by which the tube body in a deflected state is intended to extend again in the distal end direction. Accordingly, when the stent 3 is released, the tube body does not extend in the distal end direction. Therefore, the length of the stent 3 is not shortened, and thus, the stent 3 can be caused to indwell a living body in a suitable state.

After the stent 3 is released, the stent delivery system 1 and the guide wire 210 are removed via the guiding catheter 220. After the guiding catheter 220 is removed from the catheter introducer 200, the catheter introducer 200 is removed from the living body, thereby completing manual skills.

As described above, the stent delivery system 1 according to the exemplary embodiment has the extension restraining wire 9 (extension restraining shaft) which restrains the tube body from extending in the distal end direction. Accordingly, the extension restraining wire 9 can stop the force by which the tube body in a deflected state in the proximal end direction is intended to extend again in the distal end direction. When the stent 3 is released, the tube body can be restrained from extending in the distal end direction. Therefore, the stent 3 is enabled to expand and indwell in a suitable state while a phenomenon of the shortened length of the stent 3 is reduced.

It is most preferable that a portion to which the distal end 91 of the extension restraining wire 9 is fixed is the stent proximal portion latch section 22 coming into contact with the proximal surface of the stent 3. However, the portion may not necessarily be the stent proximal portion latch section 22.

It is preferable that the portion to which the distal end 91 of the extension restraining wire 9 is fixed is in the vicinity of the stent proximal portion latch section 22 of the tube body. The portion is preferably fixed within a range of 0 mm to 300 mm from the stent proximal portion latch section 22 in the proximal end direction, in the tube body moving integrally with the stent proximal portion latch section 22, more preferably within a range of 0 to 100 mm, and much more preferably within a range of 0 to 10 mm.

This range enables the extension restraining wire 9 to sufficiently achieve an advantageous effect of limiting a phenomenon of the shortened stent 3.

In addition, the stent delivery system 1 has the interlocking unit (the first gear portion 125 and the second gear portion 160) which interlocks and operates the first pulling unit 120 and the second pulling unit 130, and which can release the interlocking therebetween. Therefore, the first pulling unit 120 and the second pulling unit 130 are interlocked and operated, thereby improving workability. In a case where only the first pulling unit 120 needs to be operated, it is possible to release the interlocking therebetween.

In addition, in a state where the first pulling unit 120 and the second pulling unit 130 are interlocked by the interlocking unit (the first gear portion 125 and the second gear portion 160), the movement amount of the extension restraining wire 9 moved by the second pulling unit 130 in the proximal end direction is equal to or smaller than the movement amount of the pulling wire 6 moved by the first pulling unit 120 in the proximal end direction. Accordingly, the movement amount of the stent proximal portion latch section 22 (stent latch section) in the proximal end direction is equal to or smaller than the length in which the stent 3 is intended to move. Therefore, the stent proximal portion latch section 22 is not separated from the stent 3. When the stent 3 is released, the stent proximal portion latch section 22 can satisfactorily restricts the movement of the stent 3, and the stent 3 can be suitably released.

In addition, the first pulling unit 120 has the first movement restriction unit (the first biasing member 150 and the first gear portion 125) which restricts the movement of the pulling wire 6 in the distal end direction. The second pulling unit 130 has the second movement restriction unit (the second biasing member 170 and the second gear portion 160) which restricts the movement of the extension restraining wire 9 in the distal end direction. Accordingly, it is possible to satisfactorily maintain a state where the first pulling unit 120 and the second pulling unit 130 are moved in the proximal end direction, thereby improving operability.

Without being limited to the above-described exemplary embodiment, the present disclosure can be modified in various ways by those skilled in the art within the technical spirit of the present invention.

For example, according to the exemplary embodiment, the interlocking unit (the first gear portion 125 and the second gear portion 160) is provided, and the pulling wire 6 and the extension restraining wire 9 can be interlocked and pulled. However, without the interlocking therebetween, a roller portion which can be rotatably operated is also disposed in the second pulling unit 130. In this manner, the pulling wire 6 and the extension restraining wire 9 can be separately operated.

In this case, the second step of causing the extension restraining wire 9 to pull the tube body can be performed not only concurrently with the first step of causing the pulling wire 6 to pull the stent accommodation unit 5, but also after the first step.

In addition, the first pulling unit 120 and the second pulling unit 130 adopt a structure in which the pulling wire 6 and the extension restraining wire 9 are wound by the rotary operation. However, the winding structure may not be adopted. For example, a sliding movement structure may be adopted.

In addition, the stent delivery system 1 according to the above-described exemplary embodiment employs a so-called rapid exchange type in which a side portion on the distal side has the opening 23 into which the guide wire is inserted, but a configuration is not limited thereto. The stent delivery system 1 may employ a so-called over-the-wire type in which the guide wire lumen extends from the distal end of the proximal end of the tube body.

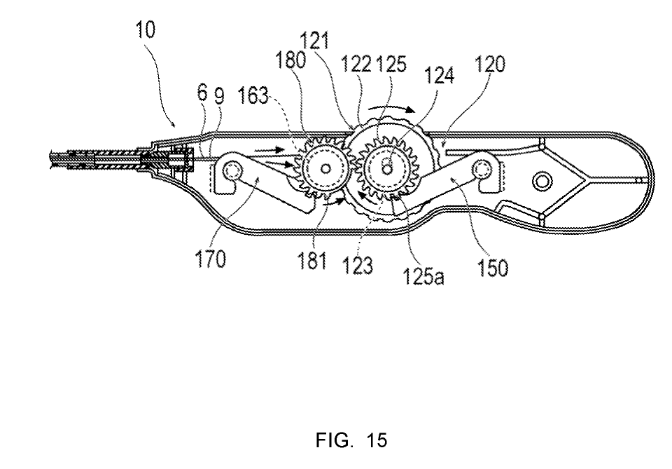

In addition, as illustrated in FIG. 15, the teeth 181 of the second gear portion 180 may be formed around less than an entire periphery of the gear portion.

In this case, the first gear portion 125 and the second gear portion 180 do not need to be structurally separated from each other. In a state where the second gear portion 180 is rotated and meshes with the first gear portion 125, the second gear portion 180 is not rotatable, thereby releasing the interlocking between the first gear portion 125 and the second gear portion 180.

In addition, the pulling shaft for pulling the stent accommodation unit may not have a wire shape. For example, the pulling shaft may be a strip-like body such as a belt, or may be a tube body which is continuously formed from the stent accommodation unit.

In addition, the extension restraining shaft which restrains the extension of the tube body may also not have the wire shape similarly to the pulling shaft.

What is claimed is:

1. A stent delivery system comprising:
a tube body which has a guide wire lumen, a stent accommodation unit which is slidable in a proximal end direction of the tube body while covering a distal side of the tube body, a substantially cylindrical stent which is expandable radially outward using a self-expandable force by being accommodated inside the stent accommodation unit in a contracted state in a central axis direction and being released from the stent accommodation unit, a pulling shaft for pulling the stent accommodation unit to the tube body in the proximal end direction by one end portion being fixed to the stent accommodation unit and being moved in the proximal end direction, and a stent latch section for restricting a movement of the stent in the proximal end direction by coming into contact with a proximal end of the stent accommodated inside the stent accommodation unit disposed in the tube body;
an extension restraining shaft having one end portion fixed to the tube body so as to restrain the tube body from extending in a distal end direction;
a first pulling unit disposed in a proximal portion of the stent delivery system, a proximal portion of the pulling shaft being interlocked with the first pulling unit so as to move the pulling shaft in the proximal end direction; and
a second pulling unit disposed in the proximal portion of the stent delivery system, a proximal portion of the extension restraining shaft being interlocked with the second pulling unit so as to move the extension restraining shaft in the proximal end direction.

2. The stent delivery system according to claim 1, further comprising:
an interlocking unit that interlocks and operates the first pulling unit and the second pulling unit, and that can release the interlocking therebetween.

3. The stent delivery system according to claim 2,
wherein in a state where the first pulling unit and the second pulling unit are interlocked with each other by the interlocking unit, a movement amount of the extension restraining shaft moved by the second pulling unit in the proximal end direction is equal to or smaller than a movement amount of the pulling shaft moved by the first pulling unit in the proximal end direction.

4. The stent delivery system according to claim 1,
wherein the first pulling unit has a first movement restriction unit that restricts the movement of the pulling shaft in the distal end direction, and
wherein the second pulling unit has a second movement restriction unit that restricts the movement of the extension restraining shaft in the distal end direction.

5. The stent delivery system according to claim 1,
wherein the stent latch section include a proximal portion latch section for latching a proximal portion of the stent and further includes a distal portion latch section for latching a distal portion of the stent.

6. The stent delivery system according to claim 5,
wherein the stent accommodation unit includes a stent accommodation portion disposed between the distal portion latch section and the proximal portion latch section, the stent being accommodated with the stent accommodation portion.

7. The stent delivery system according to claim 5,
wherein a distal end of the extension restraining shaft is fixed to the proximal portion latch section.

8. The stent delivery system according to claim 1,
further comprising a slide tube, a distal end of the slide tube closely disposed to a proximal end of the stent accommodation unit, the slide tube being moved by the pulling shaft together with the stent accommodation unit.

9. The stent delivery system according to claim 9,
wherein the slide tube includes a slide tube main body and a distal side tubular member, the distal side tubular member having a decreased diameter portion.

10. The stent delivery system according to claim 9,
wherein the decreased diameter portion is disposed between a distal end and a proximal end of the distal side tubular member, and further comprising a ring-shaped member disposed between the distal end of the slide tube main body and the decreased diameter portion.

11. The stent delivery system according to claim 10,
wherein the pulling shaft is fixed to the ring-shaped member.

12. The stent delivery system according to claim 1,
wherein the tube body includes a distal side tube and a distal member fixed to a distal end of the distal side tube, the distal member configured to prevent the stent accommodation unit from moving in the distal end direction.

13. A stent delivery method for delivering a stent by using a stent delivery system including a tube body which has a guide wire lumen, a stent accommodation unit which is slidable in a proximal end direction of the tube body while covering a distal side of the tube body, a substantially cylindrical stent which is expandable radially outward using a self-expandable force by being accommodated inside the stent accommodation unit in a contracted state in a central axis direction and being released from the stent accommodation unit, a pulling shaft for pulling the stent accommodation unit to the tube body in the proximal end direction by one end portion being fixed to the stent accommodation unit and being moved in the proximal end direction, and a stent latch section for restricting a movement of the stent in the proximal end direction by coming into contact with a proximal end of the stent accommodated inside the stent accommodation unit disposed in the tube body, the stent delivery method comprising:
a first step of causing the pulling shaft to move the stent accommodation unit in the proximal end direction, while causing the stent latch section to restrict the movement of the stent in the proximal end direction;
a second step of causing an extension restraining shaft whose one end portion is fixed to the tube body to pull the tube body in the proximal end direction concurrently with or after the first step so as to apply a tensile force to a site fixed to the tube body in the proximal end direction; and
a third step of causing the pulling shaft to move the stent accommodation unit in the proximal end direction, after the second step, and expanding the stent by using a self-expandable force after releasing the stent in a distal end direction of the stent accommodation unit, while causing the stent latch section to restrict the movement of the stent in the proximal end direction.

14. The stent delivery method according to claim 13, further comprising:
performing the second step concurrently with the first step by interlocking and operating a first pulling unit that is disposed in a proximal portion of the stent delivery system, and with which a proximal portion of the pulling shaft is interlocked so as to move the pulling shaft in the proximal end direction, and a second pulling unit that is disposed in the proximal portion of the stent delivery system, and with which a proximal portion of the extension restraining shaft is interlocked so as to move the extension restraining shaft in the proximal end direction; and
a step of releasing the interlocking between the first pulling unit and the second pulling unit after the second step.

15. The stent delivery method according to claim 14,
wherein a movement amount of the pulling shaft pulled in the proximal end direction by the first pulling unit is equal to or larger than a movement amount of the extension restraining shaft pulled in the proximal end direction by the second pulling unit.
